(19) United States
(12) Patent Application Publication
(10) Pub. No.: US 2004/0006335 A1
(43) Pub. Date: Jan. 8, 2004

(54) Cauterizing surgical saw

(76) Inventor: Lawrence L. Garrison, Gulfport, MI (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/192,827

(22) Filed: Jul. 8, 2002

(51) Int. Cl. A61B 18/08

(52) U.S. Cl. 606/27; 606/49; 606/177

(57) ABSTRACT

A surgical saw and method for cauterizing tissue, especially bone tissue, substantially immediately after the tissue is cut. The saw preferably includes a body portion defining a handle. A saw blade extends from the body portion of the saw and defines a cutting surface. A cauterizing element is fixed for movement with the saw and generally trails the cutting surface of the saw blade when the saw is moved in a cutting direction. The cauterizing element may be disposed on at least a portion of the saw blade. The saw may also comprise a blade guard and a cauterizing element may optionally be disposed on the blade guard. The cauterizing element may be activated simultaneously with activation of the saw blade.

### BACKGROUND OF THE INVENTION

1. Field of the Invention

This invention relates generally to surgical saws. More specifically, this invention relates to a surgical saw that is configured to both cut tissue and cauterize the tissue that has been cut.

2. Description of the Related Art

Surgical saws are utilized in a variety of surgical procedures where it is necessary to cut through tissue. For example, in certain types of surgery, the sternum must be cut so that the ribs may be spread apart allowing access to the organs within the chest cavity. Although the sternum is bone tissue, a significant amount of bleeding may occur from the cut edges.

Presently, surgeons utilize a surgical saw to cut the sternum. Once the sternum has been cut, a separate cautery device, such as bone wax or a cautery pencil, may be used to control the bleeding of the portion of the sternum that has been cut. However, due to the amount of time it takes to cut the entire length of the sternum, by the time the cut is finished, the initial portion of the cut may have been allowed to bleed for a significant period of time. An alternative method is to complete a portion of the cut and then stop and use the cautery device to control the bleeding. Once the bleeding has been controlled or stopped for the initial portion of the cut, the surgeon may cut another length of the sternum before stopping and repeating the procedure. This is repeated until the entire sternum is cut.

Both of the methods outlined above are inefficient and each results in an undesirable portion of the surgery duration being occupied in separately cutting the sternum with a surgical saw and then controlling the bleeding with a cautery device. It is highly desirable to reduce the amount of time occupied during surgery in performing tasks that are peripheral to the surgical procedure itself, such as cutting and cauterizing the sternum, in order to keep the overall duration of the surgery as short as possible and minimize blood loss.

### SUMMARY OF THE INVENTION

Accordingly, a need exists for more efficient cutting and cauterizing of the sternum during certain medical procedures. In accordance with the present invention, a surgical saw is provided, having a saw blade and at least one cauterizing element constructed to produce a cauterization zone proximate the saw blade that is moveable with the saw. By providing a cauterization zone that is moveable with the saw, cutting and cauterizing of tissue may be done at substantially the same time using a single instrument. This reduces the amount of bleeding in comparison to when the cutting and cauterizing are performed separately. In addition, by providing a cauterization zone that is moveable with the saw, the amount of time necessary for cutting and cauterizing is reduced to approximately only the amount of time necessary for cutting alone.

The surgical saw may include a body portion defining a handle and a saw blade coupled to the body portion. The surgical saw also includes a motor for imparting motion to the saw blade. A cauterizing element is provided and is configured to produce a cauterization area proximate the saw blade. The cauterization area is moveable with the surgical saw.

In accordance with another aspect of the invention, there is provided a method of cutting and cauterizing bone tissue. The method includes providing a surgical saw and providing a cauterizing element moveable with the surgical saw. The method further includes directing the surgical saw so as to cut the bone tissue, the cauterizing element moving with the surgical saw to cauterize the cut portion of the bone tissue.

In accordance with another aspect of the present invention, there is provided a sternotomy surgical saw. The surgical saw includes a body portion defining a handle. The saw also includes a saw blade coupled to the body portion. The saw blade includes a cutting portion having a cutting surface facing a first direction. A motor imparts a cutting motion to the saw blade and a cauterizing element is fixed for movement with the surgical saw. The cauterizing element generally trails the saw blade when the surgical saw is moved in the first direction.

The present invention also provides a method of cauterizing and cutting a sternum. The method includes providing a surgical saw having a saw blade and a motor for imparting a cutting motion to the saw blade. The method further includes directing the surgical saw to cut through the sternum. The cut portions of the sternum form two opposing surfaces defining a kerf. The method also includes providing a cauterizing element generally fixed for movement with the surgical saw and passing the cauterizing element through the kerf thereby cauterizing the opposing surfaces of the sternum.

Further features and advantages of the present invention will become apparent to those of skill in the art in view of the disclosure herein, when considered together with the attached drawings and claims.

### BRIEF DESCRIPTION OF THE DRAWINGS

These and other aspects, features, and advantages will now be described in the context of drawings of the preferred embodiments, which are intended merely to illustrate and not to limit the present invention. The drawings contain five figures in which:

FIG. 1 is a schematic illustration of a sternotomy surgical saw incorporating a cauterizing element disposed on a saw blade of the saw for cauterizing the sternum after it is cut;

FIG. 2 is an enlarged side view of the saw blade and blade guard of the surgical saw of FIG. 1. Optional cauterizing elements are disposed on various portions of the blade guard;

FIG. 3 is a front view of the saw blade and blade guard of the surgical saw of FIG. 1 taken along the view line 3-3 of FIG. 2;

FIG. 4 is a side view of a portion of the surgical saw of FIG. 1 cutting and cauterizing a sternum; and

FIG. 5 is a top plan view of an oscillating saw blade embodiment incorporating a cauterizing element.

### DETAILED DESCRIPTION OF THE PREFERRED EMBODIMENT

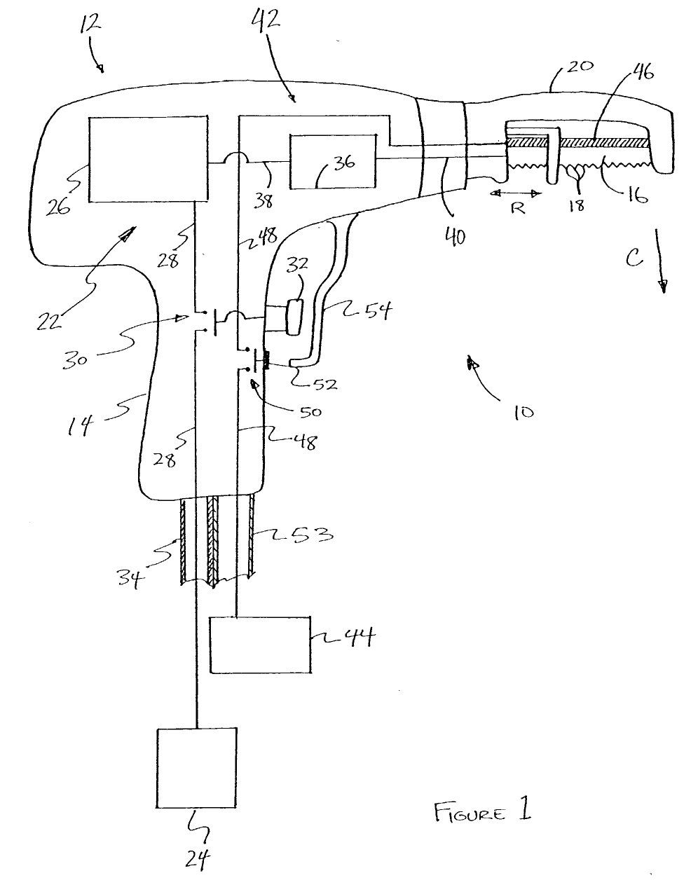

FIG. 1 is a partial schematic view of a preferred surgical saw 10 incorporating a cauterizing element for cauterizing tissue after it has been or while it is being cut. The illustrated saw 10 is a sternotomy saw suitable for cutting the sternum of a human or animal. The saw 10 generally comprises a body portion 12, which defines a handle 14 for holding the saw 10 during use. A saw blade 16 extends from the body portion 12 and includes a cutting surface defining a plurality of saw teeth 18. Preferably, the saw blade is generally rectangular in cross-section and extends generally along a longitudinal axis that is substantially transverse to the longitudinal axis of the handle 14.

Desirably, the saw 10 includes a blade guard 20 that extends from the body portion 12 in a direction generally parallel to the longitudinal axis of the saw blade 16. The blade guard 20 is positioned on a side of the saw blade 16 opposite the teeth 18 and is substantially coplanar with the saw blade 16 so as to trail the saw blade 16 when the saw 10 is moved in a cutting direction, as indicated by the arrow C. The blade guard 20 may be connected to the saw body portion 12 in any suitable manner, such as with mechanical fasteners for example.

The surgical saw 10 includes a drive arrangement 22 for imparting a cutting motion to the saw blade 16. The drive arrangement 22 comprises a power source 24 selectively connectable to a motor 26 through connection 28. A switch assembly 30 is provided to selectively connect the power source 24 and the motor 26 and is actuated by a suitable control 32, such as a trigger assembly located on the handle 14. Thus, when the trigger 32 is moved toward the handle 14, the power source 24 and the motor 26 are connected such that power is delivered to the motor 26. Alternatively, the control 32 for the drive arrangement 22 may comprise a foot pedal arrangement.

The power source 24 and the motor 26 may be of any suitable type. For example, a preferred power source 24 comprises a pneumatic pump and the motor 26 comprises a pneumatically driven motor. In such an embodiment, the connection 28 desirably comprises a conduit defined by a hose 34. In another embodiment, the motor 26 may be a hydraulic motor and the power source 24 may comprise a hydraulic pump. In still another embodiment, the motor 26 may be electrically driven and power source 24 may comprise a source of electricity, such as a battery. In this embodiment, the power source 24 may be portable and may be coupled to the body portion 12 of the surgical saw 10.

The drive arrangement 22 may include a converter 36, if necessary, to convert the drive motion of the motor 26 into the desired cutting motion of the saw blade 16. The converter 36 is coupled to the motor 26 and saw blade 16 through suitable connections 38 and 40, respectively. For example, the illustrated surgical saw 10 desirably drives the saw blade 16 in a reciprocating manner, as indicated by the arrow R of FIG. 1. That is, the saw blade 16 moves in a repeating pattern, back and forth along its longitudinal axis. In the illustrated embodiment, the converter 36 may comprise a cam assembly to convert rotational motion of the motor 26 into linear, reciprocating motion of the saw blade 16.

The surgical saw 10 additionally comprises a cautery arrangement, indicated generally by the reference numeral 42. Desirably, the cautery arrangement 42 comprises a power source 44 selectively connectable to at least one cauterizing element 46 through a suitable connection 48. A switch assembly 50 permits the selective connection of the power source 44 to the cauterizing element 46 through the connection 48. The switch 50 is desirably actuated by a suitable control 52, such as a push-button or trigger. When connected to the power source 44, the cauterizing element 46 is desirably configured to cauterize tissue that contacts, or comes in proximity to the cauterizing element 46. For example, a preferred power source 44 comprises a source of electricity. The cauterizing element 46 may comprise any of a variety of resistive heating elements well known in the electrosurgical and electrocautery arts. In this embodiment, the connection 48 may comprise electrical wire(s) housed within a conduit defined by a tube, or sheath 53. Thus, when connected to the power source 44, or source of electricity, the cauterizing element 46 desirably heats to a temperature sufficient to cauterize the desired tissue, such as the sternum bone of a human or animal. Alternatively, other suitable types of cautery arrangements 42 may be provided such as arrangements utilizing radio frequency emissions or various types of lasers, for example.

Any of a variety of heating elements may be utilized with the saw of the present invention, as will be apparent to those of skill in the art in view of the disclosure herein. For example, resistive heating elements as discussed above may be used. In a resistive heating embodiment, either the saw blade itself or one or more elements carried by or trailing the saw blade completes a driving circuit to cause a controlled heating of the element. In one embodiment, a resistive heating element in the form of a thin conductive strip is carried by each side of the saw blade, separated from the saw blade by a thin film polymeric insulator layer capable of withstanding cauterizing temperatures. The distal end of the heater elements may be soldered or otherwise electrically connected to the saw blade, so that the blade becomes the return circuit conductor.

Alternatively, a resistive heat source or other heat source may be thermally coupled to the proximal end of the saw blade, such as by wrapping a conductive coil around the saw blade or thermally coupled support, and energizing the coil to generate heat. Heat is thereafter conducted along the length of the saw blade, to provide cautery.

With any of the heat elements disclosed herein, the switch 50 may be closed to allow current to flow to or otherwise activate the heaters. Two or more heaters may be wired in parallel. In an embodiment having two resistive heaters carried by opposing sides of the blade, for example, the internal resistance of the heaters may be on the order of about 5 to 10 ohms, in one embodiment about 8 ohms, or 4 ohms per heater. The typical heater may comprise any of a variety of resistive materials, such as alumina of silicon nitride or similar glass or ceramic material. This material exhibits relatively high wattage density, rapid heat increase to 1000 degrees within one second, high level of insulation and non-stick nature of the ceramic to charred tissue. The preferred tip operating temperature range is 650 to 700° F.

A speaker, light or other indicium may be utilized to signal the surgeon when proper heater element temperature has been achieved for cauterization. The speaker will sound when the instrument reaches the preset temperature after the switch has been closed to initiate heating. The unit may also contain a temperature control. The temperature may be varied by positioning a SET/READ switch to the SET position and rotating a temperature adjust knob to the desired temperature. Alternatively, the cauterizing temperature may be preset at the point of manufacture, and not adjustable by the clinician.

A digital temperature display may be provided to report the desired set temperature and or actual temperature of the heating element(s). The temperature adjust control may either be of an analogue or digital type. This control allows the surgeon to select a temperature for a desired effect depending on the thickness and moisture content of the tissue to be cauterized. The digital temperature display may indicate the actual temperature of the heater elements when the SET/READ switch is positioned in the READ position. An LED indicator may be incorporated into the power supply, which is illuminated when the batteries are recharging. A Heater On Indicator may be incorporated into the power supply, which is illuminated when the heater elements are heated. Power may be derived from rechargeable batteries, or from connection to a 110 VAC line. A charging circuit (not shown) may be used to regulate the recharging process. See U.S. Pat. No. 6,059,781, the disclosure of which is incorporated in its entirety herein by reference. Alternative, well known heat sources such as ultrasound, microwave or laser may also be used. However, the relative cost and complexity may not be justified for the present sternotomy saw application.

As an alternative to conductive heating elements, any of a variety of electrocoagulating configurations may also be used. Electrocoagulating instruments include at least one conductive electrode. Radio frequency energy is conducted through this electrode to either a remote conductive body-plate (monopolar) or to a second, closely-spaced (e.g., on the other side of the blade) conductive electrode (bipolar). Current passing between the two electrodes will coagulate blood and other body fluids placed between them.

In the monopolar electrocautery configuration, high frequency electric current is passed from the cautery probe through the tissue to the grounding pad. The cautery probe may be the saw blade, or one or more conductive elements carried by or trailing the saw blade. Heat is generated in the tissue at the site of contact of the probe to the tissue by the flow of current through the electrical resistance of the tissue in the preferred path between the probe contact site and the grounding pad. In such devices, the current is continuous sinusoidal or amplitude modulated.

The heat generated by the cautery of monopolar electrocautery systems may not be uniform since the heating of the tissue is greater in the preferred path of current of lower resistance. For example, monopolar devices generally direct electric current along a defined path from the exposed or active electrode through the patient's body to the return electrode, which is externally attached to a suitable location on the patient. This creates the potential danger that the electric current will flow through undefined paths in the patient's body, thereby increasing the risk of unwanted electrical stimulation to portions of the patient's body. In addition, since the defined path through the patient's body has a relatively high impedance (because of the large distance or resistivity of the patient's body), large voltage differences must typically be applied between the return and active electrodes in order to generate a current suitable for ablation or cutting of the target tissue. This current, however, may inadvertently flow along body paths having less impedance than the defined electrical path, which will substantially increase the current flowing through these paths, possibly causing damage to or destroying tissue along and surrounding this pathway. As the current flows from the point of contact of the probe to the surrounding tissue, heating also tends to spread beyond the contact point of the probe to the surrounding tissue thereby potentially causing damage to the surrounding tissue.

If this is considered to be a disadvantage in the sternotomy application, it may be overcome by the use of a bipolar cautery configuration. In a bipolar embodiment, two electrodes are provided on the saw blade, such as by placing insulated thin film conductive elements on either side of the saw blade as has been discussed. Since the electrodes are insulated from the saw blade but are in electrical communication with the adjacent tissue on opposing sides of the cut, current can flow from one side of the blade to the other without the spread of current to surrounding parts of the body

Radio frequency (RF) lesion generators work on the same principle as the monopolar cautery system except that a lower level of current is used and the current is of the continuous sinusoidal form resulting in more uniform tissue destruction. However, such systems are typically used for creating lesions.

One example of a radio frequency surgical tool which may be adapted for coagulation is disclosed in U.S. Pat. No. 5,019,076, the disclosure of which is incorporated in its entirety herein by reference. The tool of U.S. Pat. No. 5,019,076 is capable of cutting and vaporizing tissue and coagulating vessels without the spread of heat to the surrounding tissue. In the disclosed device, a high frequency (13.56 or 27.0 MHz) current is passed through an amplifier, a matching network and a solenoid coil to generate an electromagnetic field. This in turn induces eddy currents in the tissue. Touching the tissue by a probe which is AC-coupled to a return circuit draws the eddy currents out of the tissue at the contact point of the probe producing intense heat which can cut and vaporize tissue as well as coagulate vessels.

Devices incorporating radio frequency electrodes for use in electrosurgical and electrocautery techniques are described in U.S. Pat. Nos. 5,281,216; 4,943,290; 4,936,301; 4,593,691; 4,228,800; and 4,202,337. U.S. Pat. Nos. 5,195,959 and 4,674,499 describe monopolar and bipolar electrosurgical devices, respectively, that include a conduit for irrigating the surgical site.

The configuration of the cautery elements can be varied, depending upon the desired clinical performance. For example, the cautery elements will generally extend at least in the plane of the cutting blade, to cauterize the opposing sides of the cut. In addition, the cautery element may extend out of the cutting plane, such as to cauterize a strip along the anterior surface and/or posterior surface of the sternum or other tissue, along the length of the cut. This may be accomplished by providing a bend in an anterior and or posterior portion of the cautery element such that it provides a degree of out of cutting plane cautery. The out of plane cautery may also be accomplished by separate, adjacent cautery elements. The out of plane angle may be varied depending upon the desired performance, but will generally be within the range of from about 45 degrees to about 135 degrees from the cutting plane, and may be approximately 90 degrees.

Regardless the cauterizing modality, cauterizing element 46 is preferably fixed for movement with the surgical saw 10. In the illustrated embodiment of FIG. 1, the cauterizing element 46 is disposed on the saw blade 16 such as on a rearward portion of the saw blade 16 such that the cauterizing element 46 trails the cutting surface of the saw blade 16 when the surgical saw 10 is moved in a cutting direction C. Desirably, the cauterizing element 46 is disposed substantially along the entire effective cutting length of the saw blade 16.

The surgical saw 10 of FIG. 1 preferably includes a master control, or trigger 54, configured to actuate both the drive arrangement control 32 and the cautery arrangement control 52 simultaneously. Thus, when the master trigger 54 is actuated, power is supplied to the motor 26 thus driving the saw blade 16 in a reciprocating motion and, simultaneously, power is supplied to the cauterizing element 46 thus heating the element 46 to a temperature sufficient to cauterize the sternum bone. Advantageously, such an arrangement allows simultaneous cutting and cauterizing of tissue, such as a sternum, as will be described in greater detail below. Other suitable master control 54 arrangements may alternatively be provided. For example, a single external control may be used to actuate both the drive arrangement 22 and the cautery arrangement 42 rather than providing separate controls 32, 52 actuated by a master control 54. A preheat cycle may be built in, to allow the cautery element to reach the desired temperature before operation of the saw, such as in a resistive heating embodiment.

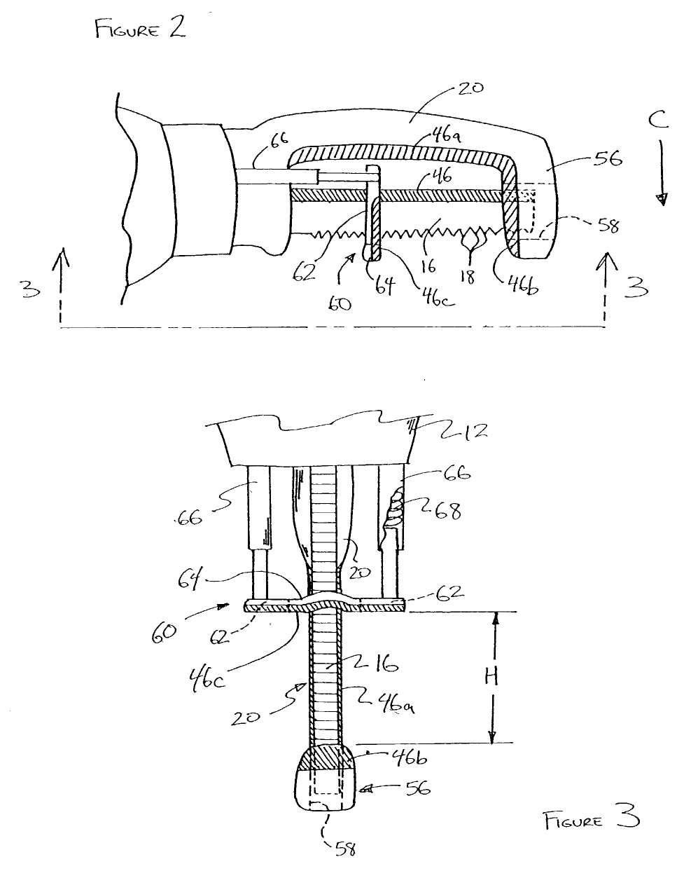

With reference to FIGS. 2 and 3, enlarged side and front views, respectively, of the saw blade 16 and blade guard 20 are illustrated. In addition to having a cauterizing element 46 on the saw blade 16, cauterizing elements may also be provided on portions of the blade guard 20. As described above, the blade guard 20 desirably extends generally parallel to the longitudinal axis of the saw blade 16. Additionally, the illustrated blade guard 20 includes a fixed end portion 56 which extends generally transverse to the longitudinal axis of the saw blade 16. The end portion 56 desirable extends toward the saw blade 16 and at least partially surrounds a distal end of the saw blade 16. In the illustrated embodiment, the end portion 56 of the blade guard 20 completely surrounds the blade 16 and defines a clearance slot 58 in which the end of the saw blade 16 is located. Desirably, the end of the saw blade 16 remains within the slot 58 during its entire length of travel while reciprocating. Such an arrangement inhibits damage to tissue other than that intended to be cut.

The illustrated blade guard 20 also includes a moveable upper transverse portion 60 extending generally in the same direction as the end portion 56. The illustrated upper portion 60 is substantially U-shaped with the side portions 62 being located on the sides of the saw blade 16 and the bottom portion 64 connecting the side portions and passing in front of the cutting surface of the saw blade 16. The upper portion 60 of the guard 20 is desirably connected to the body portion 12 of the saw 10 by a pair of telescoping slide assemblies 66. The slides 66 permit the upper portion 60 of the guard 20 to move linearly relative to the end portion 56 along an axis generally parallel to the longitudinal axis of the saw blade 16. Each of the illustrated telescoping slides 66 include a spring 68 (one shown) to bias the upper portion 60 of the guard 20 away from the body portion 12 of the surgical saw 10.

A linear distance H is defined between an upper surface of the end portion 56 of the guard 20 and a lower surface of the upper transverse portion 60 of the guard 20, as illustrated in FIG. 3. The linear distance H varies as the upper transverse portion 60 is moved (against the biasing force of the springs 68) in relation to the fixed end portion 56 of the guard 20. Thus, the linear distance H automatically adjusts to accommodate sternums of various thicknesses.

The surgical saw 10 illustrated in FIGS. 2 and 3 includes a cauterizing element on at least a portion of the blade guard 20. The cauterizing element on the guard 20 may be in addition to the element 46 of the saw blade 16, or may be provided as an alternative to placing a cauterizing element 46 on the saw blade 16. Specifically, in the illustrated embodiment, the longitudinal portion of the blade guard 20 includes a cauterizing element 46a in addition to a cauterizing element 46b on the end portion 56 of the guard 20 and a cauterizing element 46c on the upper transverse portion 60 of the blade guard 20. Cauterizing elements 46 may be provided at each of the above-described locations or provided on only some of these locations. Desirably, at least a portion of the cauterizing elements 46b and 46c are positioned rearward of the cutting teeth 18 of the saw blade 16 such that at least a portion of the elements 46b, 46c trail the cutting surface of the blade 16 when the saw 10 is moved in a cutting direction C.

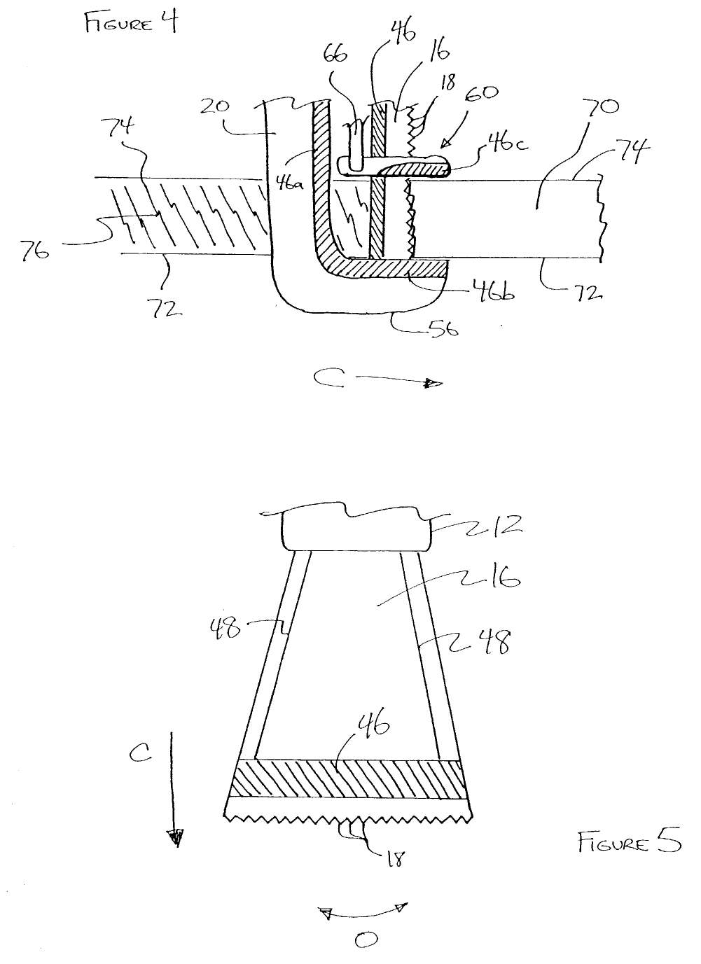

FIG. 4 illustrates a preferred embodiment of a surgical saw 10 cutting and cauterizing a sternum 70. In use, the sternum 70 is positioned between the end portion 56 and the upper transverse portion 60 of the blade guard 20. Preferably, the end portion 56 of the guard 20 is held against a lower surface 72 of the sternum 70. The slide assemblies 66 bias the upper transverse portion 60 into contact with an upper surface 74 of the sternum 70 due to the force exerted by the springs 68.

The drive arrangement control 32 is actuated to drive the saw blade 16 in a cutting motion, preferably by actuating the master control 54 such that the cautery arrangement control 52 is simultaneously actuated to heat one or more of the cauterizing elements 46, ⁴6a, 46b, 46c. Advantageously, as the saw 10 is moved in a cutting direction C thereby cutting the sternum 70, the cauterizing elements 46, 46a, 46b, 46c move with the saw 10 to cauterize the cut portions of the sternum 70.

As the sternum 70 is cut, the saw blade 16 removes material thereby creating a slot, or kerf, defined between the two opposing surfaces of the sternum 70 that have been cut, indicated by the reference numeral 76 in FIG. 4 (one shown). Although the sternum 70 is bone tissue, significant bleeding occurs when the sternum 70 is cut. Advantageously, in preferred embodiments, a cauterizing element 46, 46a follows the cutting surface of the blade 16 to cauterize the opposing surfaces 76 of the sternum 70 that have been cut. In addition, bleeding may also occur from the lower and upper surfaces 72, 74 of the sternum 70. Advantageously, the cauterizing elements 46b, 46c disposed on the end portion 56 and upper transverse portion 60, respectively, comprise at least a portion that trails the cutting edge of the blade thereby cauterizing the lower and upper surfaces 72, 74 of the sternum 70 as the saw 10 is moved in a cutting direction C. Accordingly, preferred embodiments of the sternotomy saw 10 cut and cauterize the sternum 70 with a single motion of the saw 10. Such a feature substantially reduces or eliminates the need for a separate cautery device, such as a cautery pencil when performing surgical procedures that require cutting of the sternum. As a result, bleeding of the cut portion of the sternum 70 is efficiently controlled. In addition, because cutting and cauterizing occur nearly simultaneously, the overall time required for the surgery is reduced by nearly the entire amount of time necessary to cauterize the cut portion of the sternum 70 using prior cautery devices.

In an alternative arrangement, the blade 16 and blade guard 20 may be rotated 180° with respect to the saw body 12 from the orientation of FIG. 1. Such an arrangement allows the surgeon to cut with the saw 10 using a pulling motion, rather than a pushing motion. Optionally, the saw 10 may be capable of permitting selective rotation of the blade 16 and guard 20 to permit the surgeon to select between one of the two configurations, or some point in between. Accordingly, the cutting direction C may vary with respect to the saw body 12 from the arrangement illustrated in FIG. 1. Thus, the cutting direction C is determined by the direction that the blade 16 is designed to cut, and is not limited to a specific direction with respect to the body portion 12 of the saw 10.

The principles and features described in relation to FIGS. 1-4 may also be applied to other types of surgical saws operating in a different manner, saws used to cut bones other than the sternum, or saws used to cut tissue other than bone. FIG. 5 illustrates a saw blade 16 for an oscillating saw that incorporates a cauterizing element 46 disposed on the saw blade 16. A portion of the electrical connection 48 (i.e., electrical leads) extend from the body portion 12 of the saw 10 to the cauterizing element 46 and are desirably attached to the blade 16.

The blade 16 of an oscillating saw pivots from side to side in an arcuate path covering a relatively small angle, as indicated by the arrow O in FIG. 5. The cutting direction C for an oscillating saw is generally parallel to the longitudinal axis of the saw blade 16, as illustrated in FIG. 5. Advantageously, at least a portion of the cauterizing element 46 trails the cutting surface (teeth 18) of the saw blade 16 as the saw if moved in the cutting direction C. Thus, as in the previously-described embodiment, the cauterizing element 46 moves with the saw to cauterize the cut portion of the bone, or other tissue, substantially immediately after it has been cut.

Although the present invention has been described in the context of preferred embodiments, modifications apparent to those of skill in the art are considered to be within the spirit and fair scope of the invention. For example, as discussed above, the specific locations of the illustrated cauterizing elements are merely examples and other locations may be possible without departing from the scope of the present invention. In addition, not all of the illustrated cauterizing elements or other disclosed features need to be provided in order to practice the invention. The cauterizing elements incorporated as desired in order to suit an individual application. Further, the principles disclosed in the present application may be applied to other types of saws, or other surgical instruments, to derive the benefits as taught and suggested herein. Accordingly, the scope of the present invention is to be defined only by the appended claims.

What is claimed is:

1. A surgical saw, comprising:
a body portion defining a handle;
a saw blade coupled to said body portion;
a motor for imparting motion to said saw blade; and
at least one cauterizing element configured to produce a cauterization area proximate said saw blade, said cauterization area being moveable with said surgical saw.

2. The surgical saw of claim 1, wherein said cauterization element trails a cutting edge of said saw blade when said surgical saw is moved in a cutting direction.

3. The surgical saw of claim 1, wherein at least a portion of said cauterizing element is disposed on said saw blade.

4. The surgical saw of claim 1, additionally comprising a blade guard extending from said body portion in a general direction of said saw blade and rearward thereof, wherein at least a portion of said cauterizing element is disposed on said blade guard.

5. The surgical saw of claim 4, said blade guard additionally comprising at least one portion extending generally transverse relative to said saw blade, wherein at least a portion of said cauterizing element is disposed on said transverse portion of said blade guard.

6. The surgical saw of claim 1, wherein said cauterizing element comprises an electrical resistance heater for producing heat within said cauterization area.

7. The surgical saw of claim 6, wherein said cauterizing element produces a temperature of between about 650° F. and 700° F. within said cauterization area.

8. The surgical saw of claim 1, wherein said cauterizing element is activated in conjunction with activation of said saw blade.

9. A method of cauterizing bone tissue, comprising;
providing a surgical saw;
providing a cauterizing element moveable with said surgical saw;
directing said surgical saw so as to cut said bone tissue, said cauterizing element moving with said surgical saw to cauterize said cut portion of said bone tissue.

10. The method of claim 9, wherein said surgical saw comprises a saw blade, at least a portion of said cauterizing element being provided on said saw blade.

11. The method of claim 9, wherein said surgical saw comprises a saw blade and a blade guard arranged to trail said saw blade when said surgical saw is moved in a cutting direction, at least a portion of said cauterizing element being provided on said blade guard.

12. The method of claim 9, wherein said cauterizing element is an electrocautery element.

13. The method of claim 9, additionally comprising activating said cauterizing element when said surgical saw is activated.

14. A stemotomy surgical saw, comprising:
a body portion defining a handle;
a saw blade coupled to said body portion, said saw blade comprising a cutting portion having a cutting surface facing a first direction;
a motor for imparting a cutting motion to said saw blade;
a cauterizing element fixed for movement with said surgical saw, said cauterizing element generally trailing said saw blade when said surgical saw is moved in said first direction.

15. The saw of claim 14, wherein said cauterizing element is carried by said saw blade.

16. The saw of claim 14, additionally comprising a blade guard extending from said body portion and being generally coplanar with said saw blade, said blade guard being positioned on an opposite side of said saw blade from said cutting surface, wherein at least a portion of said cauterizing element is disposed on said blade guard.

17. The saw of claim 16, wherein said blade guard additionally comprises at least a first transverse portion at least partially surrounding said saw blade, at least a portion of said cauterizing element being disposed on said first transverse portion.

18. The saw of claim 17, wherein said blade guard additionally comprises a second transverse portion positioned between said first transverse portion and said body portion, at least a portion of said cauterizing element being disposed on said second transverse portion.

19. The saw of claim 14, additionally comprising a power source for inducing a flow of electrical current through said cauterizing element.

20. A method of cutting and cauterizing a sternum, comprising:
providing a surgical saw having a saw blade and a motor for imparting a cutting motion to said saw blade;
directing said surgical saw to cut through said sternum, said cut portions of said sternum forming two opposing surfaces defining a kerf;
providing a cauterizing element generally fixed for movement with said surgical saw;
passing said cauterizing element through said kerf thereby cauterizing said opposing surfaces of said sternum.

21. The method of claim 20, additionally comprising positioning a portion of said cauterizing element both above and below said sternum so as to cauterize a portion of both an upper and lower surface of said cut portions of said sternum.

22. The method of claim 20, wherein at least a portion of said cauterizing element is disposed on said saw blade.

23. The method of claim 20, additionally comprising providing a blade guard generally trailing said saw blade during said cutting of said sternum and disposing at least a portion of said cauterizing element on said blade guard.

24. The method of claim 20, additionally comprising activating said cauterizing element simultaneously with activating said motor.
